(19) United States
(12) Patent Application Publication
(10) Pub. No.: US 2025/0345424 A1
(43) Pub. Date: Nov. 13, 2025

(54) TREATMENT OF AUTOIMMUNE DISEASES WITH ENGINEERED IMMUNE CELLS

(71) Applicant: Caribou Biosciences, Inc., Berkeley, CA (US)

(72) Inventors: Steven B. Kanner, Los Angeles, CA (US); George Kwong, San Jose, CA (US)

(73) Assignee: Caribou Biosciences, Inc., Berkeley, CA (US)

(21) Appl. No.: 18/390,978

(22) PCT Filed: Jun. 5, 2023
(86) PCT No.: PCT/US2023/067936

§ 371 Date: Dec. 20, 2023

Related U.S. Application Data
(60) Provisional application No. 63/349,286, filed on Jun. 6, 2022.
(60) Provisional application No. 63/379,564, filed on Oct. 14, 2022.

(51) Int. Cl. A61K 40/11; A61K 35/17; A61K 40/31; A61K 40/41; A61K 40/42; A61K 40/50; A61P 37/02; C07K 14/725; C12N 5/10; C12N 9/22; C12N 15/113; C12N 15/86; G01N 33/50

(52) U.S. Cl. CPC A61K 40/11; A61K 35/17; A61K 40/31; A61K 40/416; A61K 40/4211; A61K 40/50; A61P 37/02; C07K 14/7051; C12N 5/10; C12N 9/222; C12N 15/113; C12N 15/86; G01N 33/5052; A61K 2239/13; A61K 2239/21; A61K 2239/38; A61K 2239/48; C12N 2310/20; C12N 2510/00; G01N 2500/10

(57) ABSTRACT

The invention comprises methods and compositions for treating autoimmune diseases with engineered immune cells including cytotoxic T cells and natural killer (NK) cells. The engineered immune cells comprise a chimeric antigen receptor (CAR). Methods of making the engineered cells, methods of administration and treatment regimens are also disclosed.

### FIELD OF THE INVENTION

The invention relates to therapies utilizing engineered T cells expressing a chimeric antigen receptor (CAR-T cells) and more specifically, to methods of using CAR-T cells to treat autoimmune diseases.

### BACKGROUND OF THE INVENTION

Lupus and rheumatoid arthritis are two of the most prevalent autoimmune diseases, affecting an estimated 5 million and 14 million people world-wide. Lupus (systemic lupus erythematosus, SLE) affects women of childbearing age. Rheumatoid arthritis (RA) strikes both genders between ages of 35 and 50 often resulting in disability. SLE and RA are autoimmune diseases for which no cure exists, and symptoms are often inadequately managed with medication. Autoimmune disease results from abnormal activity of the immune system including B and T cells directed against “self” or autoantigens. Current treatment includes high-dose corticosteroids to effect general immunosuppression.

Lupus is characterized by the presence of B cells with antibodies against cellular nucleoproteins. Therapies developed against B cell lymphomas (B cell depleting therapy) have been successfully used to manage lupus and multiple sclerosis. These therapies include monoclonal antibodies (mAbs) targeting CD19, CD20, B cell maturation antigen (BCMA), or BAFF-R. Unfortunately, mAb therapies usually require weekly intravenous administration with beneficial effect seen at six weeks after the primary infusion. For some patients, the symptoms return nine months post-infusion.

For example, rituximab (Rituxan®) is an anti-CD20 antibody targeting B cells. It has been shown to be effective against lupus. However, unlike with the treatment of tumors, management of autoimmune disease requires repeated administrations of the therapeutic agent and over time, resistance develops.

There is a need for a more reliable and potent therapy for lupus and RA that would be well tolerated by patients.

### SUMMARY OF THE INVENTION

The invention comprises methods and compositions for treating autoimmune diseases with engineered immune cells including T cells and natural killer (NK) cells. The engineered immune cells comprise a chimeric antigen receptor (CAR). The CAR-T cells or CAR-NK cells are administered at doses much lower than the doses of the same CAR-T cells or CAR-NK cells used to treat B cell malignancies.

In one embodiment, the invention is a method of treating an autoimmune disease in a patient, the method comprising: administering to the patient an amount of a composition comprising CD19-targeting engineered immune cells, thereby improving one or more symptoms of the autoimmune disease in the patient. In some embodiments, the autoimmune disease is selected from a group consisting of: Systemic Lupus Erythematosus (SLE), Rheumatoid Arthritis (RA), Type 1 Diabetes (T1D), Sjögren's syndrome, and Multiple Sclerosis (MS). In some embodiments, the patient is a human. In some embodiments, the one or more symptoms of the autoimmune disease is selected from the group consisting of proteinuria, alopecia, increased IgM and IgG antibody titers, the presence of anti-nucleoprotein IgG or IgM in blood serum, increased B cell counts in blood plasma, and the presence of skin lesions or discoloration. In some embodiments the antibody-producing cells are B cells. In some embodiments the CD19-targeting engineered immune cells are CAR-T cells expressing an anti-CD19 chimeric antigen receptor (CAR). In some embodiments the CD19-targeting engineered immune cells are CAR-natural killer (NK) cells expressing an anti-CD19 chimeric antigen receptor (CAR). In some embodiments, the CD19-targeting engineered immune cells are allogeneic. In some embodiments of the method, the allogeneic immune cells comprise an armoring genome modification. In some embodiments, the armoring genome modification comprises inactivation of the PDCD1 gene.

In some embodiments, the anti-CD19 CAR comprises an anti-CD19 scFv, a transmembrane domain and an intracellular stimulatory domain. In some embodiments, the anti-CD19 CAR further comprises a signal peptide and a hinge. In some embodiments, the anti-CD19 CAR comprises FMC63, a CD8 hinge, a CD8 transmembrane domain, a 4-1BB co-stimulatory domain and a CD3 zeta signaling domain. In some embodiments, the anti-CD19 CAR is encoded by a nucleic acid comprising a coding sequence for the anti-CD19 CAR and a promoter. In some embodiments, the nucleic acid is integrated into the genome of the engineered immune cell. In some embodiments, the integration of the nucleic acid coding for the anti-CD19 CAR is performed using a CRISPR nuclease and a nucleic acid-targeting nucleic acid (NATNA). In some embodiments, prior to the integration, the nucleic acid coding for the anti-CD19 CAR is delivered into the immune cell via a viral vector.

In some embodiments, the amount of the composition administered to the patient comprises a dose of CD19-targeting engineered immune cells equivalent to 1/1000 of the dose used to treat B cell malignancies with the CD19-targeting engineered immune cells. In some embodiments, the amount of the composition administered to the patient comprises between 10,000 and 100,000 of the CD19-targeting engineered immune cells. In some embodiments, the amount of the composition administered to the patient comprises between 100 and 1,000 of the CD19-targeting engineered immune cells per kilogram of body weight of the patient. In some embodiments, the amount of the composition administered to the patient comprises about 40,000 of the CD19-targeting engineered immune cells. In some embodiments, the amount of the composition administered to the patient comprises about 600 of the CD19-targeting engineered immune cells per kilogram of body weight of the patient. In some embodiments, the amount of the composition administered to the patient comprises no greater than 600,000 of the CD19-targeting engineered immune cells. In some embodiments, the amount of the composition administered to the patient comprises no greater than 10,000 of the CD19-targeting engineered immune cells per kilogram of body weight of the patient.

In some embodiments, the administering is performed intravenously. In some embodiments, the administering is performed 2-4 times per year. In some embodiments, prior to the administering, the patient undergoes lymphodepletion. In some embodiments, the lymphodepletion comprises administration of a compound selected from a group consisting of cyclophosphamide, fludarabine, azathioprine, methotrexate, mycophenolate, a calcineurin inhibitor, and volcosporin. In some embodiments, the lymphodepletion comprises administering cyclophosphamide at 60 mg/kg per day for up to 2 days. In some embodiments, the lymphodepletion further comprises administering fludarabine at 25 mg/m² per day for up to 5 days.

In some embodiments, the method further comprises assessing the patient for improvements in one or more symptoms selected from the group consisting of proteinuria, alopecia, increased IgM and IgG antibody titers, the presence of anti-nucleoprotein IgG or IgM in blood serum, increased B cell counts in blood plasma, and the presence of skin lesions or discoloration. In some embodiments, the method further comprises increasing the dose of the CD19-targeting engineered immune cells administered to the patient if an improvement is not observed.

In some embodiments, the composition further comprises one or more pharmaceutically acceptable excipients. In some embodiments, the one or more excipients are selected from the group consisting of carbohydrates, inorganic salts, antimicrobial agents, antioxidants, surfactants, buffers, acids, bases, and combinations thereof. In some embodiments, the composition further comprises a freezing agent.

In one embodiment, the invention is a composition for treating an autoimmune disease comprising CD19-targeting engineered immune cells in the amount equivalent to 1/1000 of s dose used to treat B cell malignancies with the CD19-targeting engineered immune cells. In some embodiments, the autoimmune disease is selected from a group consisting of: Systemic Lupus Erythematosus (SLE), Rheumatoid Arthritis (RA), Type 1 Diabetes (T1D), Sjögren's syndrome, and Multiple Sclerosis (MS). In some embodiments, the CD19-targeting engineered immune cells are CAR-T cells expressing an anti-CD19 chimeric antigen receptor (CAR). In some embodiments, the CD19-targeting engineered immune cells are CAR-natural killer (NK) cells expressing an anti-CD19 chimeric antigen receptor (CAR). In some embodiments, the CD19-targeting engineered immune cells are allogeneic. In some embodiments of the composition, the allogeneic immune cells comprise an armoring genome modification. In some embodiments, the armoring genome modification comprises inactivation of the PDCD1 gene.

In some embodiments, the anti-CD19 CAR comprises an anti-CD19 scFv, a transmembrane domain and an intracellular stimulatory domain. In some embodiments, anti-CD19 CAR further comprises a signal peptide and a hinge. In some embodiments, the anti-CD19 CAR comprises FMC63, a CD8 hinge, a CD8 transmembrane domain, a 4-1BB co-stimulatory domain and a CD3 zeta signaling domain.

In some embodiments, the composition comprises between 10,000 and 10,000,000 of the CD19-targeting engineered immune cells. In some embodiments, the amount of the composition administered to the patient comprises between 100 and 100,000 of the CD19-targeting engineered immune cells per kilogram of body weight of the patient. In some embodiments, the amount of the composition administered to the patient comprises about 40,000 of the CD19-targeting engineered immune cells. In some embodiments, the amount of the composition administered to the patient comprises about 600 of CD19-targeting engineered immune cells per kilogram of body weight of the patient. In some embodiments, the amount of the composition administered to the patient comprises no greater than 600,000 of the CD19-targeting engineered immune cells. In some embodiments, the amount of the composition administered to the patient comprises no greater than 10,000 of the CD19-targeting engineered immune cells per kilogram of body weight of the patient. In some embodiments, the amount of the composition administered to the patient comprises no greater than 40,000,000 of the CD19-targeting engineered immune cells. In some embodiments, the amount of the composition administered to the patient comprises no greater than 60,000 of the CD19-targeting engineered immune cells per kilogram of body weight of the patient.

In some embodiments, the composition further comprises one or more pharmaceutically acceptable excipients. In some embodiments, the one or more excipients are selected from the group consisting of carbohydrates, inorganic salts, antimicrobial agents, antioxidants, surfactants, buffers, acids, bases, and combinations thereof. In some embodiments, the composition further comprises a freezing agent.

In some embodiments, the invention is a method of treating an autoimmune disease in a patient, the method comprising: administering to the patient an amount of a composition comprising engineered immune cells expressing an anti-CD19 CAR comprising FMC63, a CD8 hinge, a CD8 transmembrane domain, a 4-1BB co-stimulatory domain and a CD3 zeta signaling domain, wherein the immune cells have been assessed for in vitro activity against B cells. In some embodiments, the activity against B cells is assessed as cytotoxicity in co-culture with B cell comprising compositions. In some embodiments, the B cell comprising composition is selected from blood plasma, PBMC fraction and a B cell fraction. In some embodiments, the co-culture has an effector cell:target cell ratio between 1:10 and 10:1, e.g., between 1:8 and 8:1.

In some embodiments, the activity against B cells is assessed as reduction of antibody secretion by B cells. In some embodiments, the reduction of antibody secretion is assessed by measuring the total IgG concentration in a culture comprising B cells. In some embodiments, the culture comprising B cells is selected from blood plasma, PBMC fraction and a B cell fraction. In some embodiments, the reduction of antibody secretion is assessed by measuring the concentration of IgG characteristic of autoimmune disease in a culture comprising B cells. In some embodiments, the culture comprising B cells is selected from blood plasma, PBMC fraction and a B cell fraction.

### BRIEF DESCRIPTION OF THE DRAWINGS

FIG. 1 depicts a nucleic acid expression construct encoding an anti-CD19 chimeric antigen receptor (CAR) referred to as CB-010.

FIG. 2 is a diagram of gene editing steps used to generate the CAR-T cells “CB-010” with the CAR construct shown in FIG. 1, and the resulting phenotype of CB-010.

FIG. 3 shows results of in vitro cytotoxicity assessment of CB-010.

FIG. 4 shows results of in vitro cytotoxicity assessment of CB-010 separately for SLE-derived cellular fractions and RA-derived cellular fractions.

FIG. 5 shows measurements of autoimmune antibody concentrations in co-cultures of CB-010 with SLE-derived cellular fractions and RA-derived cellular fractions.

### DETAILED DESCRIPTION OF THE INVENTION

### Definitions

Unless defined otherwise, technical, and scientific terms used herein have the same meaning as commonly understood by a person of ordinary skill in the art. See, Sambrook et al., Molecular Cloning, A Laboratory Manual, 4^{th} Ed. Cold Spring Harbor Lab Press (2012).

The following definitions are provided to aid in understanding of the disclosure.

The term “therapeutic benefit” refers to an effect that improves the condition of the patient with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the tumor, or prevention of metastasis, or prolonging overall survival (OS) or progression free survival (PFS) of a patient with cancer.

The terms “pharmaceutically acceptable” and “pharmacologically acceptable” refer to molecular entities and compositions that do not produce an adverse, allergic, or other deleterious reaction in a patient. For example, the pharmaceutically and pharmacologically acceptable preparations should meet the standards set forth by the FDA Office of Biological Standards.

The term “pharmaceutically acceptable carrier” and “excipient” refer to aqueous solvents (e.g., water, aqueous solutions of alcohols, saline solutions, sodium chloride, Ringer's solution, etc.), non-aqueous solvents (e.g., propylene glycol, polyethylene glycol, vegetable oil, and injectable organic esters), as well as dispersion media, coatings, surfactants, gels, antioxidants, preservatives (e.g., antibacterial or antifungal agents, anti-oxidants, chelating agents, and inert gases), isotonic agents, absorption delaying agents, stabilizers, binders, disintegration agents, lubricants, sweetening agents, flavoring agents, and dyes. The concentration and pH of the various components in a pharmaceutical composition are adjusted according to well-known parameters for each component.

The term “domain” refers to one region in a polypeptide which is folded into a particular structure independently of other regions.

The term “adoptive cell” refers to a cell that can be genetically modified for use in a cell therapy treatment. Examples of adoptive cells include macrophages, and lymphocytes including T cells and natural killer (NK) cells.

The term “cell therapy” refers to the treatment of a disease or disorder that utilizes genetically modified cells. The term “adoptive cell therapy (ACT)” refers to a therapy that uses genetically modified adoptive cells. Examples of ACT include T cell therapies, CAR-T cell therapies, natural killer (NK) cell therapies and CAR-NK cell therapies.

The term “lymphocyte” refers to a leukocyte that is part of the vertebrate immune system. Lymphocytes include T cells such as CD4⁺ and/or CD8⁺ cytotoxic T cells, alpha/beta T cells, gamma/delta T cells, and regulatory T cells. Lymphocytes also include natural killer (NK) cells, natural killer T (NKT) cells, cytokine induced killer (CIK) cells, and antigen presenting cells (APCs), such as dendritic cells. Lymphocytes also include tumor infiltrating lymphocytes (TILs).

The terms “effective amount” and “therapeutically effective amount” of a composition such as a cell therapy composition, refer to a sufficient amount of the composition to provide the desired response in the patient to whom the composition is administered. In the context of administering a combination of therapeutic compounds, the effective amount of each therapeutic compound in the combination may be different from the effective amount of each therapeutic compound administered alone.

The terms “peptide,” “polypeptide,” and “protein” are interchangeable and refer to polymers of amino acids, including natural and synthetic (unnatural) amino acids, as well as amino acids not found in naturally occurring proteins, e.g., peptidomimetics, and D optical isomers. A polypeptide may be branched or linear and be interrupted by non-amino acid residues. The terms also encompass amino acid polymers that have been modified through acetylation, disulfide bond formation, glycosylation, lipidation, phosphorylation, cross-linking, or conjugation (e.g., with a label). The polypeptide need not include the full-length amino acid sequence of the reference molecule but can include only so much of the reference molecule as necessary in order for the polypeptide to retain its desired activity. For example, polypeptides comprising full-length proteins, fragments thereof, polypeptides with amino acid deletions, additions, and substitutions are encompassed by the terms “protein” and “polypeptide,” as long as the desired activity is retained. For example, polypeptides with 95%, 90%, 80%, 70% or less of sequence identity with the reference polypeptide are included as long the desired activity is retained by the polypeptides. The determination of percent identity between two nucleotide or amino acid sequences may be accomplished using a mathematical algorithm such as BLAST, NBLAST and XBLAST described in Altschul, et al. (1990, J. Mol. Biol. 215:403-410) and available from the National Center for Biotechnology Information (NCBI).

The terms “CRISPR” (clustered regularly interspaced short palindromic repeats), “CRISPR-Cas” (CRISPR-associated protein) and “CRISPR system” refer to the genome editing tool derived from prokaryotic organisms and comprising a nucleic acid guide molecule and a sequence-specific nucleic acid-guided endonuclease capable of cleaving a target nucleic acid strand at a site complementary to a sequence in the nucleic acid guide.

The term “NATNA” (nucleic acid targeting nucleic acid) refers to a nucleic acid guide molecule of the CRISPR system. NATNA may be comprised two nucleic acid targeting polynucleotides (“dual guide”) including a CRISPR RNA (crRNA) and transactivating CRISPR RNA (tracrRNA). NATNA may be comprised a single nucleic acid targeting polynucleotide (“single guide”) comprising crRNA and tracrRNA connected by a fusion region (linker). The crRNA may comprise a targeting region and an activating region. The tracrRNA may comprise a region capable of hybridizing to the activating region of the crRNA. The term “targeting region” refers to a region that is capable of hybridizing to a sequence in a target nucleic acid. The term “activating region” refers to a region that interacts with a polypeptide, e.g., a CRISPR nuclease.

B cells producing autoantibodies are at least one documented cause of autoimmune diseases such as lupus (SLE and other forms of lupus), rheumatoid arthritis (RA), Type 1 Diabetes (T1D), Sjögren's syndrome, and Multiple Sclerosis (MS). A common characteristic of active B cells is surface expression of CD19. Anti-CD19 cytotoxic T cells including autologous and allogeneic CAR-T cell therapies have been shown to effectively reduce the numbers of CD19-expressing malignant B cells in patients. Attempts to attack autoimmune B cells with CAR-T cells in the mouse model have been described in U.S. application Pub. No. US20180264038 Chimeric antigen receptor (CAR) T cells as therapeutic interventions for auto- and alloimmunity, U.S. application Pub. No. US2020078403 Use of chimeric antigen receptor modified cells to treat autoimmune disease, and U.S. application Pub. No. US20200085871 Methods of using cytotoxic T cells for treatment of autoimmune diseases.

The present invention describes the use of a low-dose of well-tolerated anti-CD19 allogeneic CAR-T cells to manage the symptoms of autoimmune disease in humans.

In some embodiments, the invention comprises adoptive cells and the use of adoptive cells to treat or alleviate autoimmune diseases including lupus, rheumatoid arthritis, Type 1 Diabetes (T1D), Sjögren's syndrome, and Multiple Sclerosis (MS). Adoptive cells of the instant invention include lymphocytes, such as T cells, CAR-T cells, NK cells, iPSC-derived NK (iNK) cells, and CAR-NK cells.

In some embodiments, the invention utilizes T cells isolated from a healthy donor. In some embodiments, the T cells are obtained from a blood sample of a healthy donor via leukapheresis. Techniques for isolating lymphocytes are well known in the art, see, e.g., Smith, J. W. (1997) Apheresis techniques and cellular immunomodulation, Ther. Apher. 1:203-206. In some embodiments, the invention utilizes a T cell composition depleted of CD4⁺ T cells (T-helper cells) known to contribute to the symptoms of autoimmune disease. In some embodiments, the invention utilizes a T cell composition substantially free of CD4⁺ T cells.

In some embodiments, the invention utilizes natural killer (NK) cells isolated from a healthy donor, e.g., from peripheral blood mononuclear cells (PBMC), leukapheresis products (PBSC), bone marrow, or umbilical cord blood by methods well known in the art, see, e.g., Spanholtz, J. et al., (2011) Clinical-grade generation of active NK cells from cord blood hematopoietic progenitor cells for immunotherapy using a closed-system culture process, PloS one, 6(6), e20740, and Shah, N., et al., (2013) Antigen presenting cell-mediated expansion of human umbilical cord blood yields log-scale expansion of natural killer cells with anti-myeloma activity. PloS one, 8(10), e76781.

In some embodiments, the invention utilizes NK cells obtained by differentiating human embryonic stem cells (hESCs) or induced pluripotency stem cells (iPSCs). NKs differentiated from iPSCs are referred to as iNK cells.

In some embodiments, the NK cells are heterologous and are haplotype-matched for the patient in one or more HLA locus, one or more KIR locus or both.

In some embodiments, the isolated NK cell composition is depleted of CD3⁺ cells. In some embodiments, the isolated NK cell composition is enriched for CD56⁺ cells. In some embodiments, the isolated NK cell composition is enriched for CD45⁺ cells. In some embodiments, the isolated cell NK composition is enriched for CD56⁺/CD45⁺ cells. In some embodiments, a quality control measure or characterization step is applied to the isolated NK cell composition, e.g., determining the percentage of CD56⁺/CD3⁻, CD45⁺/CD3⁻cells, CD56⁺/CD45⁺, or CD56⁺/CD45⁺/CD3⁻ in the composition. In some embodiments, the invention utilizes an NK cell composition substantially free of CD3⁺ cells.

In some embodiments, isolated lymphocytes are characterized in terms of specificity, frequency of each subtype, and function. In some embodiments, the isolated lymphocyte population is enriched for specific subsets of T cells, such as CD8⁺, CD25⁺, or CD62L⁺. See, e.g., Wang et al., Mol. Therapy—Oncolytics (2016) 3:16015. In some embodiments, the isolated NK cell composition is enriched for CD56⁺/CD45⁺ cells.

In some embodiments, the quality control measure or characterization step is applied to the cell-containing composition. In some embodiments, the quality control measure or characterization step is determining the percentage of CD56⁺/CD45⁺ cells in the composition by flow cytometry.

In some embodiments, after isolation, lymphocytes are activated in order to promote proliferation and differentiation into specialized lymphocytes. For example, T cells can be activated using soluble CD3/28 activators, or magnetic beads coated with anti-CD3/anti-CD28 monoclonal antibodies.

In some embodiments, the invention is a method of treating an autoimmune disease in a patient comprising administering to the patient a composition comprising immune cells expressing a CD19-targeting protein. In some embodiments, the immune cell is selected from a T cell, a natural killer (NK) cell, an iNK cell. In some embodiments, the immune cell is selected from a CAR-T cell, a CAR-NK cell.

In some embodiments, the CD19-targeting protein is an anti-CD19 T cell receptor. In some embodiments, the anti-CD19 T cell receptor in a chimeric antigen receptor (CAR). In some embodiments, the immune cells are CAR-T cells or CAR-NK cells.

In some embodiments, the CAR comprises an extracellular domain comprising an CD19-binding region, a transmembrane domain and one or more intracellular co-activation (co-stimulatory) and activation (stimulatory) domains.

In some embodiments, the CD19-binding region of the CAR is derived from a monoclonal antibody. In some embodiments, the CD19-binding region comprises a fragment of the variable portion of the heavy chain (V_{H}) or a fragment of the variable portion of the light chain (V_{L}) of a single-chain variable fragment (scFv) or a camelid single domain antibody (V_{HH}). These fragments may be derived from a monoclonal antibody. The single-chain variable fragment (scFv) has the ability to bind CD19. The scFv is comprised of the Fv regions of immunoglobulin heavy chain (H chain) and light chain (L chain) linked via a spacer sequence. In some embodiments, the CD19-binding scFv is FMC63, see Nicholson et al., (1997) Construction and characterization of a functional CD19 specific single chain Fv fragment for immunotherapy of B lineage leukaemia and lymphoma, Mol. Immunol. 34:1157.

In some embodiments, the transmembrane domain of the CAR is derived from a membrane-bound or transmembrane protein. For example, the transmembrane domain of the CAR may be the transmembrane domain of a T cell receptor alpha-chain or beta-chain, a CD3-zeta chain, CD28, CD3-epsilon chain, CD2, CD45, CD4, CD5, CD8, CD9, CD16, CD22, CD33, CD37, CD64, CD80, CD86, CD134, CD137, ICOS, CD154, DNAM1, NKp44, NKp46, NKG2D, 2B4, or GITR. In some embodiments, the transmembrane domain of the CAR is the CD8 transmembrane domain. In some embodiments, the transmembrane domain of the CAR is the CD8A transmembrane domain

The intracellular signaling domain of a CAR is responsible for activation of one or more effector functions of the immune cell expressing the CAR. In some embodiments, the intracellular signaling domain of the CAR comprises a part of or the entire sequence of the CD3-zeta chain, CD3-epsilon chain, CD2, CD28, CD27, OX40/CD134, 4-1BB/CD137, ICOS/CD278, IL-2Rbeta/CD122, IL-2Ralpha/CD132, DAP10, DAP12, DNAM1, TLR1, TLR2, TLR4, TLR5, TLR6, MyD88, CD40 or a combination thereof. In some embodiments the intracellular domain of the CAR consists of 4-1BB and CD3 zeta chain.

In some embodiments, the CAR comprises a hinge domain. In some embodiments the hinge domain of the CAR is the CD8 hinge domain. In some embodiments the hinge domain of the CAR is the CD8A hinge domain.

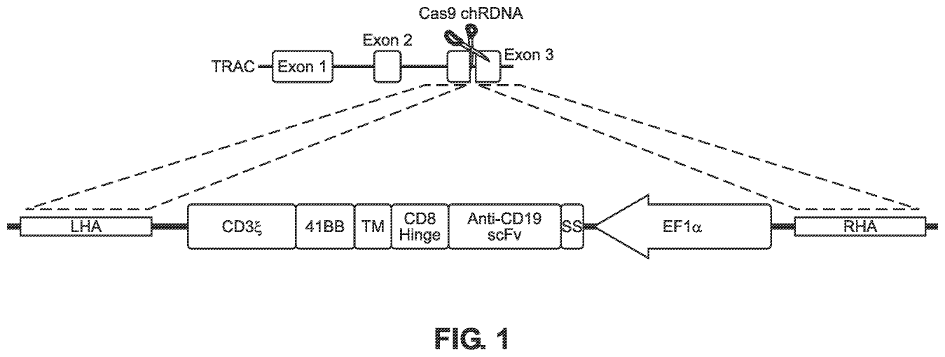

An exemplary anti-CD19 CAR is shown in FIG. 1. The CAR comprises a signal sequence, an anti CD19 scFv, a CD8 hinge domain, a transmembrane domain, a 4-1BB and CD3-zeta intracellular domains.

In some embodiments, the CAR is a fully human protein or is humanized to reduce immunogenicity in human patients. In some embodiments, the nucleic acid sequence encoding the CAR is optimized for codon usage in human cells.

The nucleic acid encoding the CAR may be introduced into a cell as a genomic DNA sequence or a cDNA sequence. The cDNA sequence comprises the open reading frame for the translation of the CAR and in some embodiments, further comprises untranslated elements that improve for example, the stability or the rate of translation of the CAR mRNA.

In some embodiments, the cell used to treat autoimmune disease (T cell, a natural killer (NK) cell, an iNK cell, a CAR-T cell, or a CAR-NK cell) further comprises a genome modification resulting in armoring of the cell against an attack by the immune system of a recipient autoimmune disease patient. In some embodiments, the armoring modification comprises protection from recognition by the cytotoxic T cells of the host. Cytotoxic T cells recognize MHC Class I antigen. MHC Class I molecule is comprised of beta-2 microglobulin (B2M) associated with heavy chains of HLA-I proteins (selected from HLA-A, HLA-B, HLA-C, HLA-E, HLA-F and HLA-G) on the surface of the cell. The B2M/HLA-I complex on the surface of the allogeneic cell is recognized by cytotoxic CD8⁺ T cells and if HLA-I is recognized as non-self, the allogeneic cell is killed by the T cells. In some embodiments, the cells of the invention comprise an armoring genomic modification comprising a disruption of the B2M gene and therefore, disruption of the MHC Class I antigen recognition and cytotoxic T cell attack.

In some embodiments, the armoring genome modification comprises disruption of recognition by the NK cells of the host. NK cells recognize cells without MHC-I protein as “missing self” and kill such cells. NK cells are inhibited by HLA-I molecules, including HLA-E, a minimally polymorphic HLA-I protein. In some embodiments, the cells of the invention comprise a first armoring genomic modification comprising a disruption of the B2M gene and therefore, disruption of the MHC Class I antigen recognition and cytotoxic T cell attack, and further comprise a second armoring genomic modification comprising an insertion of an HLA-E gene fused to beta-2-microglobulin (B2M) gene, and therefore, expression of the HLA-E/B2M construct and cloaking the cells from an attack by NK cells. See, e.g., Gornalusse et al., (2017) HLA-E-expressing pluripotent stem cells escape allogeneic responses and lysis by NK cells, Nat. Biotechnol. (2017) 35:765-772.

In some embodiments, the armoring modification comprises transcriptionally silencing or disrupting one or more immune checkpoint gene. In some embodiments, the one or more immune checkpoint gene is selected from PD1 (encoded by the PDCD1 gene), CTLA-4, LAG3, Tim3, BTLA, BY55, TIGIT, B7H5, LAIR1, SIGLEC10, and 2B4 as disclosed in the U.S. application publication US20150017136 Methods for engineering allogeneic and highly active T cell for immunotherapy.

In some embodiments, the patient receiving the treatment with immune cells expressing a CD19-targeting protein is monitored to assess the clinical manifestations of the autoimmune disease. The symptoms are expected to diminish with treatment described herein. In some embodiments, the patient is assessed for clinical manifestations of the autoimmune disease prior to the administration of the immune cells expressing a CD19-targeting protein. In some embodiments, the patient is assessed hourly, daily, weekly, or monthly after the first administration of the T cell expressing a CD19-targeting protein. In some embodiments, the patient is assessed in connection with a daily, weekly, or monthly regimen of administration of immune cells expressing a CD19-targeting protein.

In some embodiments, the clinical manifestations of the autoimmune disease include one or more of proteinuria, alopecia, organ enlargement, the presence of hypercellular glomeruli, IgG tissue deposits, IgM and IgG antibody titers and IgG or IgM antinuclear antibody in blood serum, an increase in the total number or concentration of B cells in the blood plasma, and the presence of skin lesions or discoloration. Accordingly, the patient is assessed for the clinical manifestations of the autoimmune disease by one or more of urine analysis, blood analysis (including total blood count), and physical inspection.

In some embodiments, the total number or concentration of B cells in the blood plasma is assessed by flow cytometry. In some embodiments, the presence of the IgG or IgM antinuclear antibody in blood serum is assessed by ELISA.

In some embodiments, the patient is assessed for the presence and relative number of immune cells expressing a CD19-targeting protein, such as T cells, NK cells, CAR-T cells, or CAR-NK cells. In some embodiments, the presence and relative number of the cells is assessed by one or more methods selected from flow cytometry, ELISA, fluorescent microscopy, fluorescent in situ hybridization (FISH), PCR and RT-PCR aimed at detecting the presence of the CD19-targeting protein, the gene encoding the CD19-targeting protein, or the mRNA encoding the CD19-targeting protein respectively.

In some embodiments, the anti-CD19 CAR is encoded by a nucleic acid construct introduced into the cell used to treat autoimmune disease (T cell, a natural killer (NK) cell, or an iNK cell). In some embodiments, the anti-CD19 CAR expression construct comprises a coding sequence for the CD19-targeting CAR, and a promoter.

In some embodiments, the CD19-targeting CAR expression construct is introduced via an expression vector or an RNA encoding the CD19-targeting CAR protein. In some embodiments, the target cells are contacted with the nucleic acid encoding the CD19-targeting CAR in vitro, in vivo or ex vivo.

In some embodiments, the vector is a viral vector (e.g., a retroviral vector, adenoviral vector, adeno-associated viral vector, or lentiviral vector). Suitable vectors are non-replicating in the target cells. In some embodiments, the vector is selected from or designed based on SV40, EBV, HSV, or BPV. The vector incorporates the protein expression sequences. In some embodiments, the expression sequences are codon-optimized for expression in mammalian cells. In some embodiments, the vector also incorporates regulatory sequences including transcriptional activator binding sequences, transcriptional repressor binding sequences, enhancers, introns, and the like. In some embodiments, the viral vector supplies a constitutive or an inducible promoter. In some embodiments, the promoter is selected from EF1α, PGK1, MND, Ubc, CAG, CaMKIIa, and β-Actin promoter. In some embodiments, the promoter is selected from the SV40 early and late promoters, the cytomegalovirus (CMV) immediate early promoter, and the Rous sarcoma virus long terminal repeat (RSV-LTR) promoter, mouse mammary tumor virus long terminal repeat (MMTV-LTR) promoter, the β-interferon promoter, the hsp70 promoter and EF-1α promoter. In some embodiments, the promoter is an EF-1α promoter. In some embodiments, the promoter is an MND promoter.

In some embodiments, the viral vector supplies a transcription terminator or a polyadenylation signal. In some embodiments, the transcription terminator or polyadenylation signal is the BGH transcription terminator and polyadenylation signal.

In some embodiments, the vector is a plasmid selected from a prokaryotic plasmid, a eukaryotic plasmid, and a shuttle plasmid.

In some embodiments, the expression vector comprises one or more selection marker. In some embodiments, the selection markers are antibiotic resistance genes or other negative selection markers. In some embodiments, the selection markers comprise proteins whose mRNA is transcribed together with the CD19-targeting CAR mRNA and the polycistronic transcript is cleaved prior to translation.

In some embodiments, the expression vector comprises polyadenylation sites. In some embodiments, the polyadenylation sites are SV-40 polyadenylation sites.

In some embodiments, the coding sequence of the CD19-targeting CAR is introduced into the cells via a viral vector, such as e.g., AAV vector (AAV6) or any other suitable viral vector capable of delivering an adequate payload. In some embodiments, to facilitate homologous recombination, the coding sequence is joined to homology arms located 5′ (upstream or left) and 3′ (downstream or right) of the insertion site in the desired insertion site in the genome. In some embodiments, the homology arms are about 500 bp long. In some embodiments, the sequence coding for the CD19-targeting CAR together with the homology arms are cloned into a viral vector plasmid. The plasmid is used to package the sequences into a virus.

An exemplary nucleic acid construct is shown in FIG. 1. In addition to the CAR coding region, the construct comprises an EF1α promoter, left homology arm (LHA) and a right homology arm (RHA).

In some embodiments, the cell (T cell, a natural killer (NK) cell, or an iNK cell) is contacted with a viral vector so that the genetic material delivered by the vector is integrated into the genome of the target cell and then expressed in the cell or on the cell surface. Transduced and transfected cells can be tested for transgene expression using methods well known in the art such as fluorescence-activated cell sorting (FACS), microfluidics-based screening, ELISA, or Western blot.

In some embodiments, the coding sequence for the CD19-targeting CAR is introduced into the cell (T cell, a natural killer (NK) cell, or an iNK cell) as “naked” nucleic acid by electroporation as described e.g., in U.S. Pat. No. 6,410,319.

In some embodiments, an engineered CRISPR system is introduced into the cell (T cell, a natural killer (NK) cell, or an iNK cell). In some embodiments, the CRISPR system comprises a nucleic acid-guided endonuclease and nucleic acid-targeting nucleic acid (NATNA) guides (e.g., a CRISPR guide RNAs selected from tracrRNA, crRNA or a single guide RNA incorporating the elements of the tracrRNA and crRNA in a single molecule).

In some embodiments, NATNA is selected from the embodiments described in U.S. Pat. No. 9,260,752. Briefly, a NATNA can comprise, in the order of 5′ to 3′, a spacer extension, a spacer, a minimum CRISPR repeat, a single guide connector, a minimum tracrRNA, a 3′ tracrRNA sequence, and a tracrRNA extension. In some instances, a nucleic acid-targeting nucleic acid can comprise, a tracrRNA extension, a 3′ tracrRNA sequence, a minimum tracrRNA, a single guide connector, a minimum CRISPR repeat, a spacer, and a spacer extension in any order.

In some embodiments, the guide nucleic acid-targeting nucleic acid can comprise a single guide NATNA. The NATNA comprises a spacer sequence which can be engineered to hybridize to the target nucleic acid sequence. The NATNA further comprises a CRISPR repeat comprising a sequence that can hybridize to a tracrRNA sequence. Optionally, NATNA can have a spacer extension and a tracrRNA extension. These elements can include elements that can contribute to stability of NATNA. The CRISPR repeat and the tracrRNA sequence can interact, to form a base-paired, double-stranded structure. The structure can facilitate binding of the endonuclease to the NATNA.

In some embodiments, the single guide NATNA comprises a spacer sequence located 5′ of a first duplex which comprises a region of hybridization between a minimum CRISPR repeat and minimum tracrRNA sequence. The first duplex can be interrupted by a bulge. The bulge facilitates recruitment of the endonuclease to the NATNA. The bulge can be followed by a first stem comprising a linker connecting the minimum CRISPR repeat and the minimum tracrRNA sequence. The last paired nucleotide at the 3′ end of the first duplex can be connected to a second linker connecting the first duplex to a mid-tracrRNA. The mid-tracrRNA can comprise one or more additional hairpins.

In some embodiments, the NATNA can comprise a double guide nucleic acid structure. The double guide NATNA comprises a spacer extension, a spacer, a minimum CRISPR repeat, a minimum tracrRNA sequence, a 3′ tracrRNA sequence, and a tracrRNA extension. The double guide NATNA does not include the single guide connector. Instead, the minimum CRISPR repeat sequence comprises a 3′ CRISPR repeat sequence and the minimum tracrRNA sequence comprises a 5′ tracrRNA sequence and the double guide NATNAs can hybridize via the minimum CRISPR repeat and the minimum tracrRNA sequence.

In some embodiments, NATNA is an engineered guide RNA comprising one or more DNA residues (CRISPR hybrid RDNA or chRDNA). In some embodiments, NATNA is selected from the embodiments described in U.S. Pat. No. 9,650,617. Briefly, some chRDNA for use with a Type II CRISPR system may be composed of two strands forming a secondary structure that includes an activating region composed of an upper duplex region, a lower duplex region, a bulge, a targeting region, a nexus, and one or more hairpins. A nucleotide sequence immediately downstream of a targeting region may comprise various proportions of DNA and RNA. Other chRDNA may be a single guide D(R)NA for use with a Type II CRISPR system comprising a targeting region, and an activating region composed of and a lower duplex region, an upper duplex region, a fusion region, a bulge, a nexus, and one or more hairpins. A nucleotide sequence immediately downstream of a targeting region may comprise various proportions of DNA and RNA. For example, the targeting region may comprise DNA or a mixture of DNA and RNA, and an activating region may comprise RNA or a mixture of DNA and RNA.

In some embodiments, the components of the CRISPR system are introduced into the cell in the form of nucleic acids. In some embodiments, the components of the CRISPR system are introduced into the cell in the form of DNA coding for the nucleic acid-guided endonuclease and NATNA guides. In some embodiments, the gene coding for the nucleic acid-guided endonuclease (e.g., a CRISPR nuclease selected from Cas9 and Cas12a) is inserted into a plasmid capable of propagating in the cell. In some embodiments, the gene coding for the NATNA guides is inserted into a plasmid capable of propagating in the cell.

In some embodiments, the components of the CRISPR system, i.e., the nucleic acid-guided endonuclease and NATNA guides are introduced into the cell in the form of RNA, e.g., the mRNA coding for the nucleic acid-guided endonuclease along with the NATNA guides.

In some embodiments, the components of the CRISPR system, i.e., the nucleic acid-guided endonuclease and the NATNA guides are introduced into the cell as a preassembled nucleoprotein complex. In some embodiments, the components of the CRISPR system, i.e., the nucleic acid-guided endonuclease and the NATNA guides are introduced into the cell via any combination of different means, e.g., the endonuclease is introduced as the DNA via a plasmid containing the gene encoding the endonuclease while the guides are introduced in its final format as RNA (or RNA containing DNA nucleotides).

In some embodiments, the components of the CRISPR system, i.e., the nucleic acids encoding the nucleic acid-guided endonuclease and NATNA guides are introduced into the cell via electroporation.

In some embodiments, the components of the CRISPR system, i.e., the nucleic acids coding for the nucleic acid-guided endonuclease are introduced into the cell in the form of mRNA as described e.g., in the U.S. Pat. No. 10,584,352 via electroporation of viral pseudo-transduction as described therein.

In some embodiments, the coding sequence for the CD19-targeting CAR is inserted into a double-strand break in the genome of the cell (T cell, a natural killer (NK) cell, or an iNK cell). In some embodiments, the introduction of the coding sequence coincides with inactivation of another gene by the insertion of the CAR gene (gene knock-out and simultaneous gene knock-in). In some embodiments, the insertion site and an inactivated gene is TRAC, CBLB, PDCD1, CTLA-4, LAG3, Tim3, BTLA, BY55, TIGIT, B7H5, LAIR1, SIGLEC10, and 2B4. In some embodiments, the CD19-targeting CAR sequence is inserted into the T cell receptor alpha (TRAC) gene.

In some embodiments, the CD19-targeting (anti-CD19) CAR-T cells are allogeneic. The allogeneic CAR-T cells may comprise an armoring modification protecting the allogeneic cells from an attack by the patient's (recipient's) immune system. In some embodiments, the armoring modification comprises transcriptionally silencing or disrupting one or more immune checkpoint genes. In some embodiments, the checkpoint gene is PDCD1 (encoding the PD-1 protein).

Programmed cell death protein 1 (PD-1, encoded by the gene PDCD1), also known as CD279, is a cell surface receptor that plays an important role in downregulating the immune system, and promoting self-tolerance by suppressing T cell inflammatory activity. PD-1 binds to its cognate ligand, “programmed death-ligand 1,” also known as PD-L1, CD274, and B7 homolog 1 (B7-H1) or its other ligand PD-L2, also known as CD273. PD-1 guards against autoimmunity through a dual mechanism of promoting programmed cell death (apoptosis) in antigen-specific T cells in lymph nodes, while simultaneously reducing apoptosis in anti-inflammatory, suppressive T cells (regulatory T cells). Through these mechanisms, PD-1 binding of PD-L1 inhibits the immune system, thus preventing autoimmune disorders, but also prevents the immune system from killing cancer cells. Accordingly, mutating or knocking out expression of PD-1 (e.g., by disrupting the PDCD1 gene) can be beneficial in T cell therapies.

In some embodiments, the immune checkpoint gene is disrupted using an endonuclease that specifically cleaves nucleic acid strands within a target sequence of the gene to be disrupted. The strand cleavage by the sequence-specific endonuclease results in nucleic acid strand breaks that may be repaired by non-homologous end joining (NHEJ). NHEJ is an imperfect repair process that may result in direct re-ligation but more often, results in deletion, insertion, or substitution of one or more nucleotides in the target sequence. Such deletions, insertions, or substitutions of one or more nucleotides in the target sequence may result in missense or nonsense mutations in the protein coding sequence and eliminate production of any protein or cause production of a non-functional protein.

In some embodiments, the armoring modification comprises targeted cleavage and repair of the PDCD1 gene resulting in gene inactivation. In some embodiments, the PDCD1 gene is disrupted by cleavage of the PDCD1 locus in exon 2 of the PDCD1 gene on human chromosome 2 by a CRISPR-Cas endonuclease (e.g., Cas9) and a guide polynucleotide (NATNA). In some embodiments, the guide polynucleotide (NATNA) is a CRISPR hybrid RNA-DNA polynucleotide (chRDNA).

In some embodiments, the anti-CD19 CAR-T cells are assessed for their activity against B cells. In some embodiments, the anti-CD19 CAR-T cells are assessed for their activity against B cells derived from patients diagnosed with autoimmune disease.

In some embodiments, the activity of the anti-CD19 CAR-T cells against B cells is assessed in vitro as cytotoxicity against B cells.

In some embodiments, the in vitro assessment of cytotoxic properties of anti-CD19 CAR-T cells utilizes target cells or target cell lines. In some embodiments, the target cells are primary cells obtained from human blood samples. In some embodiments, the human samples are from patients diagnosed with autoimmune disease. In some embodiments, the human samples are control samples obtained from subjects free from autoimmune disease. In some embodiments, the samples are processed to extract blood fractions such as peripheral blood mononuclear cells (PBMCs), B cells or non-B cells.

In some embodiments, target cells are established lymphoid cell lines. In some embodiments, target cells are established B cell lines. In some embodiments, target cells are established lymphoid tumor cell lines of B cell tumor cell lines.

In some embodiments, expression of CD19 in target cells is confirmed prior to assessing cytotoxicity of the anti-CD19 CAR-T cells. In some embodiments, expression of CD19 is confirmed by a method selected from flow cytometry with anti-CD19 antibody, staining with a label-conjugated anti-CD19 antibody, fluorescent in situ hybridization, Western blot or any other method known in the art to detect expression of a protein on the cell surface.

In some embodiments, cytotoxicity of the anti-CD19 CAR-T cells is assessed as lysis of B cells in vitro. The B cell lysis may be assessed by co-culturing the anti-CD19 CAR-T cells (effector cells or effectors) with a cell population comprising B cells or consisting of B cells. The co-culture may be established at different effector:target ratios (E:T ratios). In some embodiments, the E:T ratios are in the range of about 0.1:1 (1:10) to about 10:1. In some embodiments, two or more E:T ratios in the selected range are evaluated. In some embodiments, two or more or all of the E:T ratios selected from 0.125:1 (1:8), 0.25:1 (1:4), 0.5:1 (1:2), 1:1, 2:1, 4:1, 8:1 are evaluated.

In some embodiments, cell lysis is detected by labeling target cells with cell permeant stable fluorescent dyes (e.g., CellTrace™ Violet (CTV), ThermoFisher Scientific, Carlsbad, Cal.) in conjunction with viability dyes to measure specific lysis by flow cytometry. Cytotoxicity can also be determined by utilizing target cells expressing luciferase in cocultures with effector cells and measuring bioluminescence. Time lapse imaging can also be used to determine cell lysis by either incorporating a viability dye and measuring increase in fluorescence or by utilizing cells containing a fluorescent reporter and measuring decrease in fluorescence. Impedance-based systems like the xCELLigence system (Agilent, Santa Clara, Cal.) can also provide dynamic real time monitoring of cell lysis.

In some embodiments, a control experiment is performed assessing lysis of cell populations consisting of non-B cells by the anti-CD19 CAR-T cells. In some embodiments, a control experiment is performed assessing lysis of cell populations comprising both B cells and non-B cells (e.g., PBMCs) by the anti-CD19 CAR-T cells.

In some embodiments, B cell lysis by the by the anti-CD19 CAR-T cells is compared in primary cell samples from autoimmune patients and primary cell samples from subjects free from autoimmune disease.

In some embodiments, the anti-CD19 CAR-T cell population effecting the highest percentage of B cell lysis is selected for administration to a patient suffering from autoimmune disease. In some embodiments, the anti-CD19 CAR-T cell population effecting a high percentage of B cell lysis but having low non-B cell lysis is selected for administration to a patient suffering from autoimmune disease.

In some embodiments, the activity of the anti-CD19 CAR-T cells against B cells is assessed in vitro as a decrease in autoantibody secretion by the B cells. In some embodiments, autoantibody secretion is assessed by co-culturing anti-CD19 CAR-T cell (effectors, E) with a cell population comprising B cells (targets, T). In some embodiments, the co-culture is at E:T ratio in the range of about 1:10 to about 10:1. In some embodiments, the co-culture is at E:T ratio of about 1:1. In some embodiments, the autoantibodies in the co-culture supernatant are assessed qualitatively or quantitatively. The autoantibodies can be assessed as total IgG in the supernatant. Specific species of autoantibodies (e.g., anti-dsDNA IgG characteristic of SLE) can be detected with an antibody-based or antibody conjugate-based assay such as Western blotting or ELISA and similar secondary antibody-based methods with colorimetric, chemiluminescent, or fluorescent detection methods. Anti-dsDNA antibodies, can also be detected using Farr radioimmunoassay, which measures radiolabeled dsDNA bound to anti-dsDNA antibodies, or using Crithidia luciliae indirect immunofluorescence test (CLIFT).

In some embodiments, the invention comprises compositions including cells (T cells, natural killer (NK) cells, or iNK cells) expressing a CD19-targeting protein. In some embodiments, the composition comprises cytotoxic CAR-T cells or CAR-NK cells expressing an anti-CD19 chimeric antigen receptor (CAR). In some embodiments, the compositions include the cells, and one or more pharmaceutically acceptable excipients. Exemplary excipients include, without limitation, carbohydrates, inorganic salts, antimicrobial agents, antioxidants, surfactants, buffers, acids, bases, and combinations thereof. Excipients suitable for injectable compositions include water, alcohols, polyols, glycerin, vegetable oils, phospholipids, and surfactants. A carbohydrate such as a sugar, a derivatized sugar such as an alditol, aldonic acid, an esterified sugar, and/or a sugar polymer may be present as an excipient. Specific carbohydrate excipients include, for example, monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient can also include an inorganic salt or buffer such as citric acid, sodium chloride, potassium chloride, sodium sulfate, potassium nitrate, sodium phosphate monobasic, sodium phosphate dibasic, and combinations thereof.

In some embodiments, the composition further comprises an antimicrobial agent for preventing or deterring microbial growth. In some embodiments, the antimicrobial agent is selected from benzalkonium chloride, benzethonium chloride, benzyl alcohol, cetylpyridinium chloride, chlorobutanol, phenol, phenylethyl alcohol, phenylmercuric nitrate, thimerosal, and combinations thereof.

In some embodiments, the composition further comprises an antioxidant added to prevent the deterioration of the lymphocytes. In some embodiments, the antioxidant is selected from ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

In some embodiments, the composition further comprises a surfactant. In some embodiments, the surfactant is selected from polysorbates, sorbitan esters, lipids, such as phospholipids (lecithin and other phosphatidylcholines), phosphatidylethanolamines, fatty acids and fatty esters; steroids, such as cholesterol.

In some embodiments, the composition further comprises a freezing agent such as 3% to 12% dimethylsulfoxide (DMSO) or 1% to 5% human albumin.

The number of adoptive cells, such as T cells, NK cells, CAR-T cells or CAR-NK cells, in the composition will vary depending on a number of factors but will optimally be a therapeutically effective dose per vial.

A minimum or optimal therapeutically effective dose can be determined experimentally by repeated administration of increasing amounts of the composition in order to determine which amount produces a reduction in symptoms of autoimmune disease.

A maximum or optimal therapeutically effective dose can be determined experimentally by repeated administration of decreasing amounts of the composition in order to determine which amount produces a reduction in symptoms of autoimmune disease while not producing undesirable side effects or producing an acceptable degree of undesirable side effects.

The invention includes a step of administering to the patient a composition comprising immune cells (T cells, NK cells or iNK cells) expressing a CD19-targeting protein.

In some embodiments, prior to administration of the immune cells, the patient undergoes a lymphodepletion pre-treatment to reduce any immune system attack against the administered immune cells. In some embodiments, the patient is pre-treated with an immunosuppressor known to be safe and effective against autoimmune disease, see e.g. Fava A., and Petri, M. (2019) Systemic lupus erythematosus: diagnosis and clinical management, J. Autoimmun. 96:1-13.

In some embodiments, the immunosuppressor is cyclophosphamide, an alkylating agent with a history of use in lupus patients and known to deplete T and B cells.

In some embodiments, the immunosuppressor is azathioprine, a purine analogue with a history of use in lupus patients.

In some embodiments, the immunosuppressor is methothrexate, an antimetabolite with a history of use in lupus patients and known to suppress proinflammatory T cells.

In some embodiments, the immunosuppressor is mycophenolate, an agent depleting guanoside nucleotides and having a history of use in lupus patients and known to inhibit proliferation of T and B cells.

In some embodiments, the immunosuppressor is a calcineuring inhibitor (e.g., volcosporin) with a history of use in lupus patients and known to reduce T cell activity.

In some embodiments, the lymphodepletion comprises of a cyclophosphamide regimen. In some embodiments, the lymphodepletion includes administration of cyclophosphamide at 60 mg/kg per day for 2 days.

In some embodiments, the lymphodepletion further comprises fludarabine regimen. In some embodiments, the lymphodepletion includes administration of fludarabine at 25 mg/m² per day for 5 days.

At the end of the lymphodepletion pre-treatment, the patient is administered a composition including no greater than 600,000 (equivalent to no greater than 10⁴/kg) of immune cells expressing an anti-CD19 CAR. In some embodiments, the patient is administered 40,000 (equivalent to 600/kg) of anti-CD19 allogeneic CAR-T cells.

The dose of CD19-targeting cells (such as anti-CD19 CAR-T cells and CAR-NK cells) cells needed to treat autoimmune disease is substantially lower than the dose of the CAR-T or CAR-NK cells needed to treat tumors. In addition, the dose of allogeneic CAR-T or CAR-NK cells needed to achieve a therapeutic effect on tumors is substantially lower than the dose of autologous CAR-T or CAR-NK cells. Table 1 lists the relative doses of autologous anti-CD19 CAR-T cells YESCARTA®, BREYANZI® and KYMRIAH® compared to an experimental allogeneic anti-CD19 CAR-T cell composition CB-010, while CB-010 has produced a greater overall response and complete response in patients (source: Abstract for European Hematology Association (EHA), 12 May 2022 CB-010 Clinical Program Update).

TABLE 1
Dose comparison among allogeneic and
autologous CAR-T cell treatments

| | | Dose | | Dose rel. |
|---|---|---|---|---|
| Therapy | Type | Total cell # | cells/kg | CB-010 |
| CB-010 | allogeneic | 4 × 10⁷ | 6 × 10⁵ | n/a |
| YESCARTA ® | autologous | 1.2 × 10⁸ | 2 × 10⁶ | 3x greater |
| BREYANZI ® | autologous | 5 × 10⁷- | 8 × 10⁵- | 1.25-3x greater |
| | | 1.1 × 10⁸ | 1.8 × 10⁶ | |
| KYMRIAH ® | autologous | 6 × 10⁷- | 10⁶-10⁷ | 1.3-13x greater |
| | | 6 × 10⁸ | | |

### Source: Package Inserts

Mackensen et al., have achieved remission in SLE patients treated with autologous anti-CD19 CAR-T cells administered at the dose of 10⁶ cells/kg (4×10⁷-9×10⁷ cells per patient). Mackensen et al., (2022) Anti-CD19 CAR T cell therapy for refractory system lupus erythematosus, Nat. Med. 28:2124. This dose is in the range of 2-10 fold lower than the dose used to treat B cell malignancies with autologous CAR-T cells (Table 1).

It has also been demonstrated that administration of allogeneic CAR-T cells generated a better response in patients than administration of autologous CAR-T cells. Table 2 lists efficacy (measured as overall response (ORR) and complete response (CR)) produced by autologous anti-CD19 CAR-T cells YESCARTA®, BREYANZI® and KYMRIAH® compared to an experimental allogeneic anti-CD19 CAR-T cell composition CB-010 (source: EHA Abstract 12 May 2022 supra).

TABLE 2
Efficacy of allogeneic and autologous CAR-T cell treatments

| Therapy | Type | ORR, % | CR, % |
|---|---|---|---|
| CB-010 | allogeneic | 100 | 80 |
| YESCARTA ® | autologous | 73 | 52 |
| BREYANZI ® | autologous | 75 | 54 |
| KYMRIAH ® | autologous | 50 | 32 |

It has further been demonstrated that administration of allogeneic CAR-T cells produced fewer side effects than administration of autologous CAR-T cells. Table 3 lists side effects (≥grade 3 of each: cytokine release syndrome (CRS), immune effector cell-associated neurotoxicity syndrome (ICANS), and infections) produced by autologous anti-CD19 CAR-T cells YESCARTA®, BREYANZI® and KYMRIAH® compared to an experimental allogeneic anti-CD19 CAR-T cell composition CB-010 (source: EHA Abstract 12 May 2022 supra).

TABLE 3
Safety of allogeneic and autologous CAR-T cell treatments

| Therapy | Type | CRS, % | ICANS, % | infections, % |
|---|---|---|---|---|
| CB-010 | allogeneic | none | 17 | 17 |
| YESCARTA ® | autologous | 4 | 25 | 17 |
| BREYANZI ® | autologous | 9 | 12 | 19 |
| KYMRIAH ® | autologous | 23 | 23 | 40 |

In some embodiments, the dose of anti-CD19 CAR expressing cells administered to a human patient to treat autoimmune disease is about 0.1% ( 1/1000^{th}) of the dose of the same CAR-T cells administered to treat tumors. For example, for CB-010 allogeneic anti-CD19 CAR-T cells, the dose is about 4×10⁴ (40,000) of CAR-T cells compared to 4×10⁷ of CAR-T cells used to treat B cell non-Hodgkin lymphomas. Expressed in cells per kilogram of body weight, the dose is about 6×10² (600) allogeneic CAR-T cells/kg compared to 6×10³ CAR-T cells/kg used to treat B cell non-Hodgkin lymphomas. In some embodiments, the patient is administered no greater than 600,000 (equivalent to no greater than 10⁴/kg) of allogeneic anti-CD19 CAR expressing cells.

In some embodiments, the dose of anti-CD19 CAR expressing cells administered to the human patient to treat autoimmune disease is about the same as the dose of the same CAR-T cells administered to treat tumors. For example, for CB-010 allogeneic anti-CD19 CAR-T cells, the dose is about 4×10⁷ (40,000,000) total CAR-T cells. Expressed in cells per kilogram of body weight, the dose is about 6×10⁵ (60,000) allogeneic CAR-T cells/kg.

In some embodiments, the invention is method of treating an autoimmune disease in a patient comprising administering to the patient a composition comprising of cells expressing and anti-CD19 protein (such as anti-CD19 CAR-T cells or CAR-NK cells) at a dose of 10,000-100,000 cells, equivalent to about 100-1000 cells per kilogram of body weight.

In some embodiments, the invention is method of treating an autoimmune disease in a patient comprising administering to the patient a composition comprising 40,000 (equivalent to 600/kg) of anti-CD19 allogeneic CAR-T cells.

In some embodiments, the invention is method of treating an autoimmune disease in a patient comprising administering to the patient a composition comprising no greater than 600,000 (equivalent to no greater than 10⁴/kg) of anti-CD19 allogeneic CAR-T cells.

In some embodiments, the invention comprises administering to the patient the anti-CD19 allogeneic CAR-T cells at a frequency of 2-4 times per year.

In some embodiments the patient is treated with anti-CD19 allogeneic CAR-T cells more or less frequently than 2-4 times per year based on the symptom assessment described herein including blood and urine analysis, and visual assessment to detect the progress of treatment and any side effects.

In some embodiments, the therapeutic composition is administered to a patient by a route selected from intravenous, parenteral, intrathecal, local, and intramuscular. In some embodiments the administration is by infusion and the infusion is selected from a single sustained dose, a prolonged continuous infusion, and multiple infusions.

### EXAMPLES

### Example 1. (Prophetic) Administering the Anti-CD19 Allogeneic CAR-T Cells to Measurably Alleviate the Symptoms of Lupus

In this example, a human patient is subjected to one or more of urine analysis, blood analysis (including total blood count), physical assessment and is diagnosed with lupus if one or more of the following is present: proteinuria, alopecia, organ enlargement, the presence of hypercellular glomeruli, IgG tissue deposits, IgM and IgG antibody titers and IgG or IgM antinuclear antibody in blood serum, an increase in the total number or concentration of B cells in the blood plasma, and the presence of skin lesions or discoloration.

The patient undergoes lymphodepletion pre-treatment consisting of cyclophosphamide at 60 mg/kg per day for 2 days and fludarabine at 25 mg/m² per day for 5 days.

At the end of the lymphodepletion pre-treatment, the patient is administered a composition including 40,000 (equivalent to 600/kg) of anti-CD19 allogeneic CAR-T cells.

Starting with one week post-administration the patient is assessed by one or more of urine analysis, blood analysis (including total blood count), and physical assessment to detect any diminution of previously existing symptoms of lupus selected from proteinuria, alopecia, organ enlargement, the presence of hypercellular glomeruli, IgG tissue deposits, IgM and IgG antibody titers and IgG or IgM antinuclear antibody in blood serum, an increase in the total number or concentration of B cells in the blood plasma, and the presence of skin lesions or discoloration.

The total number or concentration of B cells in the blood plasma is assessed by flow cytometry. The IgG or IgM antinuclear antibody in blood serum is assessed by ELISA.

The patient is also assessed for presence (persistence) of the anti-CD19 allogeneic CAR-T cells. These cells are detected by flow cytometry, ELISA, fluorescent microscopy, fluorescent in situ hybridization (FISH), PCR and RT-PCR aimed at detecting the presence of the CD19-targeting CAR, the gene encoding the CAR, or the mRNA encoding the CAR.

If no diminution of the symptoms is observed, the patient is administered another dose or a greater dose of the anti-CD19 allogeneic CAR-T cells. If a low number or none of the anti-CD19 allogeneic CAR-T cells are detected in the patient's circulation, the patient is administered another dose or a greater dose of the anti-CD19 allogeneic CAR-T cells.

### Example 2. CB-010: Allogeneic Anti-CD19 CAR-T Cells

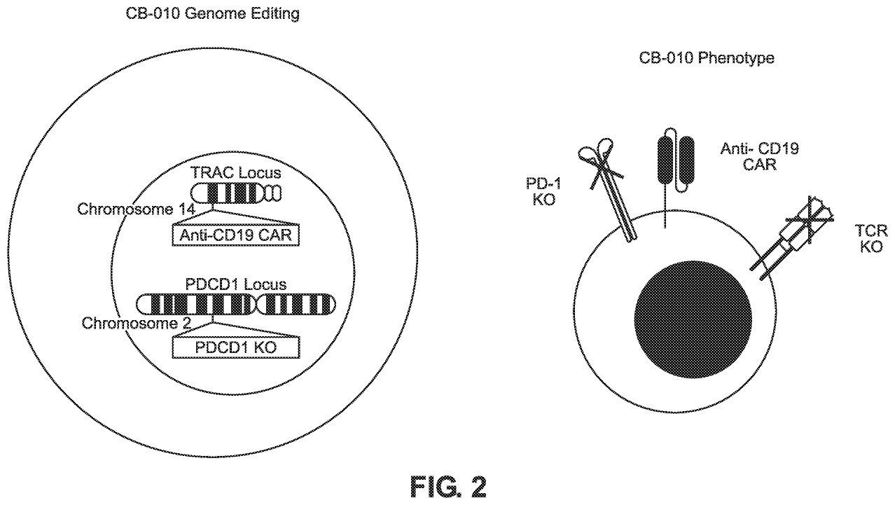

The allogeneic anti-CD19 CAR-T cells with PD-1 inactivation referred to as CB-010 (FIG. 2) were developed for relapsed/refractory B cell non-Hodgkin's lymphoma. (See Abstract for European Hematology Association (EHA), 12 May 2022 CB-010 Clinical Program Update). The structure of the CAR is shown in FIG. 1.

Briefly, the CB-010 cells were generated from T cells obtained by leukapheresis of healthy donor blood samples. CRISPR Cas9 endonuclease with chRDNAs (CRISPR hybrid RNA-DNA guides) was used for genome editing. The anti-CD19 CAR transgene (FIG. 1) was delivered via an AAV vector and inserted into the T cell receptor alpha chain (TRAC) locus on chromosome 14. Additionally, the PDCD1 gene on chromosome 2 was disrupted using Cas9/chRDNAs resulting in abrogation of PD-1 expression.

### Example 3. Specific Lysis of B Cells by the Anti-CD19 CAR-T Cells (CB-010)

In this example, the anti-CD19 CAR-T cells (allogeneic anti-CD19 CAR-T cells with PDCD1 gene inactivation referred to as CB-010 described in Abstract for European Hematology Association (EHA), 12 May 2022 CB-010 Clinical Program Update) were cocultured with cellular fractions obtained from blood samples of autoimmune patients or with isolated non-diseased B cells. As a control, donor-matched T cells with inactivated TRAC locus but no CAR insertion (TRAC KO) were used. Briefly, targets were labeled with CTV to distinguish them from effector cells. Non-diseased B cells were cocultured at the following E:T ratios: 8:1, 4:1, 2:1, 1:1, 0.5:1 0.25:1, 0.125:1, 0:1. Autoimmune patient-derived cellular fractions were cocultured at the following E:T ratios: 0.5:1, 0.25:1, 0.125:1, 0.0625:1, 0.03125:1 0.015625:1, 0.0078125:1, 0:1. Cocultures were maintained for 24 hours, after which cocultures were stained with a B cell marker-specific antibody (such as CD19 or CD20) and with a viability dye (such as propidium iodide (PI)) for cytotoxicity measurement through flow cytometry (iQue Screener Plus, Intellicyt, Albuquerque, N.M.). Cytotoxicity was determined by gating on the live cell population within the CTV-labeled target cell populations, or within B cell and non-B cell populations of the CTV-labeled target cells. Specific lysis was calculated using the following equation for each well: Specific lysis=1−(% of live target cells in coculture sample/% of live target cells in target-only sample). Specific lysis curves were then be generated for different samples, and area under the curve (AUC) measurements of specific lysis was determined for the different populations and conditions.

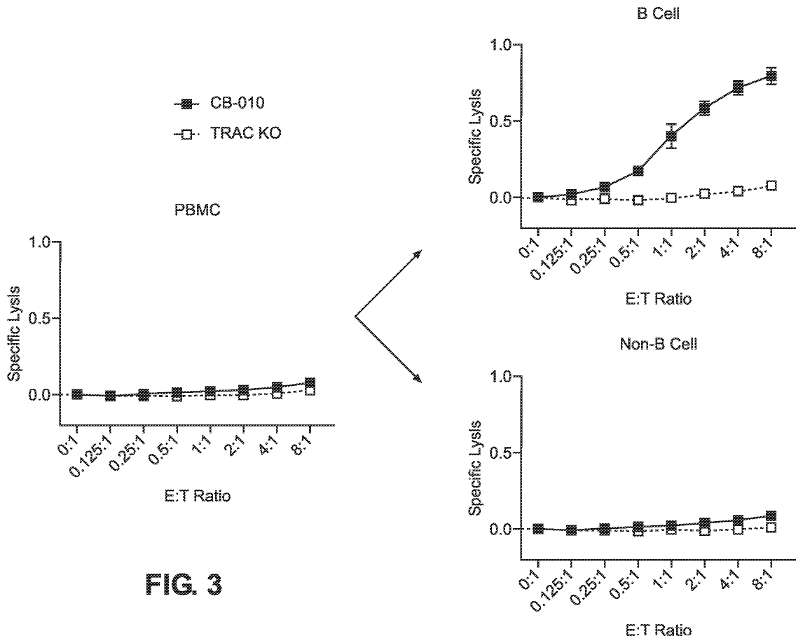

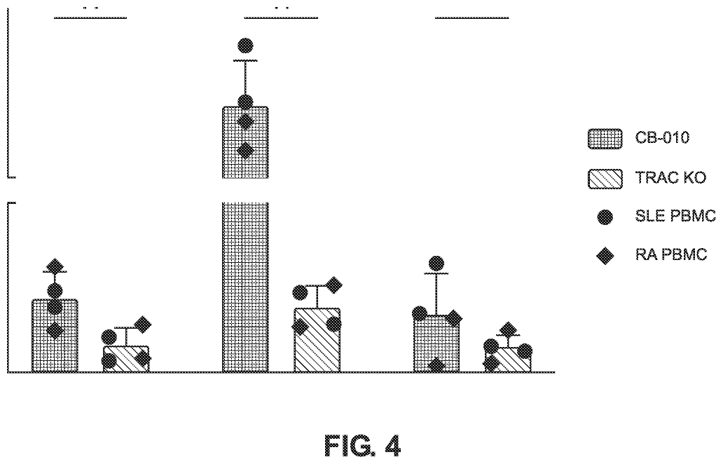

Results are shown in FIG. 3 and FIG. 4. FIG. 3 shows results of in vitro cytotoxicity assessment of CB-010 allogeneic anti-CD19 CAR-T cells. Specific lysis of PBMCs, B cells and non-B cells from Systemic Lupus Erythematosus (SLE)-derived cellular fractions at various E:T ratios with CB-010 is shown. FIG. 4 shows results of in vitro cytotoxicity assessment of CB-010 separately for SLE-derived cellular fractions and Rheumatoid Arthritis (RA)-derived cellular fractions. Cytotoxicity is expressed as the area under the curve (AUC) measurement of specific lysis for PBMCs, B cells and non-B cells from SLE patients and RA patients by CB-010. Data represents 4 independent donors (2 SLE patient-derived PBMCs and 2 RA patient-derived PBMCs). Error bars represent average±SD. ns (not significant) indicates p>0.05 and ** indicates p≤0.01 by paired t-test between CB-010 and TRAC KO coculture conditions.

### Example 4. Decrease in Autoantibody Secretion by B Cells in the Presence of the Anti-CD19 CAR-T Cells (CB-010)

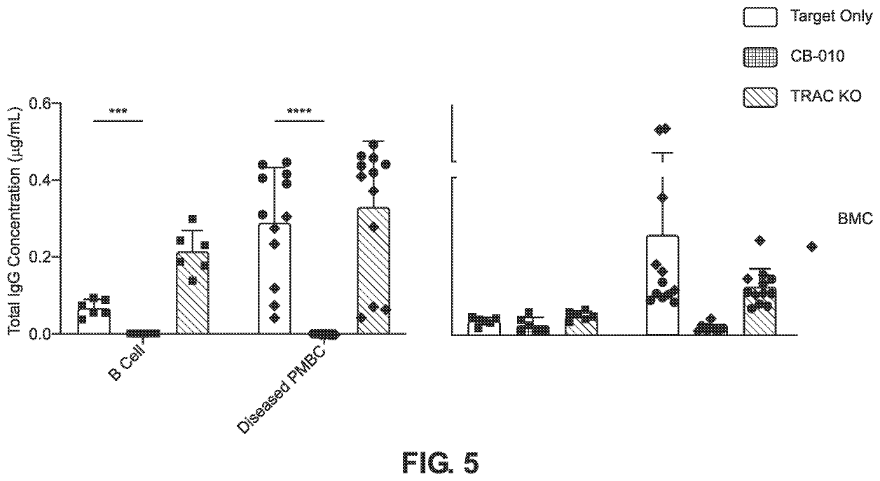

In this example, the CB-010 allogeneic anti-CD19 CAR-T cells were cocultured with cellular fractions obtained from blood samples of autoimmune patients or with isolated non-diseased B cells. As controls, targets were also cultured alone or co-cultured with donor-matched T cells with inactivated TRAC locus but no CAR insertion (TRAC KO). Non-diseased B cells were co-cultured with effector cells at a 1:1 E:T ratio, and autoimmune-derived cellular fractions were co-cultured with effector cells at a 1:4 E:T ratio to account for B cells being only a fraction of the PBMCs. Co-cultures were maintained for 6 days in the presence of ODN2006, a CpG oligonucleotide that strongly activates B cells through TLR9 activation. After 6 days, supernatants were harvested from the cocultures. Total IgG and anti-dsDNA IgG concentration were measured in the co-culture supernatants using ELISA kits specific for total IgG detection (Invitrogen, Carlsbad, Cal.) or anti-dsDNA IgG detection (Abnova, Taipei City, Taiwan). Results are shown in FIG. 5 as measurements of autoimmune antibody concentrations in co-cultures of CB-010 with SLE-derived cellular fractions and RA-derived cellular fractions. Data represents 6 independent donors (2 isolated healthy B cells, 2 SLE patient-derived PBMCs, and 2 RA patient-derived PBMCs). Error bars represent average±SD. ns (not significant) indicates p>0.05, * indicates p≤0.05, *** indicates p≤0.001, and **** indicates p≤0.0001 by paired t-test between target only and CB-010 coculture conditions.

While the invention has been described in detail with reference to specific examples, it will be apparent to one skilled in the art that various modifications can be made within the scope of this invention. Thus, the scope of the invention should not be limited by the examples described herein, but by the claims presented below.

1. A method of treating an autoimmune disease in a patient, the method comprising:
administering to the patient an amount of a composition comprising CD19-targeting engineered allogeneic CAR-T cells comprising:
an anti-CD-19 CAR wherein the anti-CD19 CAR comprises FMC63, a CD8 hinge, a CD8 transmembrane domain, a 4-1BB co-stimulatory domain and a CD3 zeta signaling domain, and further comprising an inactivated PDCD1 gene, thereby improving one or more symptoms of the autoimmune disease in the patient.

2. The method of claim 1, wherein the patient is a human, and the autoimmune disease is selected from a group consisting of: Systemic Lupus Erythematosus (SLE), Rheumatoid Arthritis (RA), Type 1 Diabetes (T1D), Sjögren's syndrome, and Multiple Sclerosis (MS).

3. (canceled)

4. The method of claim 1, wherein the one or more symptoms of the autoimmune disease is selected from the group consisting of proteinuria, alopecia, increased IgM and IgG antibody titers, the presence of anti-nucleoprotein IgG or IgM in blood serum, increased B cell counts in blood plasma, and the presence of skin lesions or discoloration.

5-13. (canceled)

14. The method of claim 1, wherein the anti-CD19 CAR is encoded by a nucleic acid comprising a coding sequence for the anti-CD19 CAR and a promoter.

15. The method of claim 14, wherein the nucleic acid is integrated into the genome of the engineered immune cell and the integration of the nucleic acid coding for the anti-CD19 CAR is performed using a CRISPR nuclease and a nucleic acid-targeting nucleic acid (NATNA).

16. (canceled)

17. The method of claim 15, wherein prior to the integration, the nucleic acid coding for the anti-CD19 CAR is delivered into the immune cell via a viral vector.

18. The method of claim 1, wherein the amount of the composition administered to the patient comprises a dose of CD19-targeting engineered immune cells equivalent to 1/1000 of the dose used to treat B cell malignancies with the CD19-targeting engineered immune cells.

19. The method of claim 1, wherein the amount of the composition administered to the patient comprises between 10,000 and 100,000,000 of the CD19-targeting engineered immune cells.

20-26. (canceled)

27. The method of claim 1, wherein prior to the administering, the patient undergoes lymphodepletion comprising administration of a compound selected from a group consisting of cyclophosphamide, fludarabine, azathioprine, methotrexate, mycophenolate, a calcineurin inhibitor, and volcosporin.

28. (canceled)

29. The method of claim 27, wherein the lymphodepletion comprises administering cyclophosphamide at 60 mg/kg per day for up to 2 days and further comprises administering fludarabine at 25 mg/m² per day for up to 5 days.

30. (canceled)

31. The method of claim 1 further comprising assessing the patient for improvements in one or more symptoms selected from the group consisting of proteinuria, alopecia, increased IgM and IgG antibody titers, the presence of anti-nucleoprotein IgG or IgM in blood serum, increased B cell counts in blood plasma, and the presence of skin lesions or discoloration.

32-35. (canceled)

36. A composition for treating an autoimmune disease comprising CD19-targeting allogeneic engineered CAR-T cells expressing a CAR comprising FMC63, a CD8 hinge, a CD8 transmembrane domain, a 4-1BB co-stimulatory domain and a CD3 zeta signaling domain, and further comprising an armoring genome modification comprising an inactivated PDCD1 gene, the cells being in the amount equivalent to 1/1000 of s dose used to treat B cell malignancies with the CD19-targeting engineered immune cells.

37. The composition of claim 36, wherein the autoimmune disease is selected from a group consisting of: Systemic Lupus Erythematosus (SLE), Rheumatoid Arthritis (RA), Type 1 Diabetes (T1D), Sjögren's syndrome, and Multiple Sclerosis (MS).

38-45. (canceled)

46. The composition of claim 36 comprising between 10,000 and 100,000 of the CD19-targeting engineered immune cells.

47-51. (canceled)

52. The composition of claim 36 further comprising one or more pharmaceutically acceptable excipients.

53-54. (canceled)

55. A method of treating an autoimmune disease in a patient, the method comprising:
administering to the patient an amount of a composition comprising engineered allogeneic immune cells expressing an anti-CD19 CAR comprising FMC63, a CD8 hinge, a CD8 transmembrane domain, a 4-1BB co-stimulatory domain, and a CD3 zeta signaling domain, wherein the immune cells have been assessed for in vitro activity against B cells.

56. The method of claim 55, wherein the activity against B cells is assessed as cytotoxicity in co-culture with B cell comprising compositions selected from blood plasma, PBMC fraction and a B cell fraction.

57. (canceled)

58. The method of claim 55, wherein the co-culture has an effector cell:target cell ratio between 1:10 and 10:1.

59. (canceled)

60. The method of claim 55, wherein the activity against B cells is assessed as reduction of antibody secretion by B cells.

61. The method of claim 60, wherein the reduction of antibody secretion is assessed by measuring the total IgG concentration in a culture comprising B cells selected from blood plasma, PBMC fraction and a B cell fraction.

62. (canceled)

63. The method of claim 60, wherein the reduction of antibody secretion is assessed by measuring the concentration of IgG characteristic of autoimmune disease in a culture comprising B cells selected from blood plasma, PBMC fraction and a B cell fraction.

64. (canceled)
